(19) United States
(12) Patent Application Publication
(10) Pub. No.: US 2024/0085365 A1
(43) Pub. Date: Mar. 14, 2024

(54) SENSORS

(71) Applicant: BRAMBLE ENERGY LIMITED, Crawley (GB)

(72) Inventor: Thomas James Mason, Crowborough (GB)

(21) Appl. No.: 18/262,815

(22) PCT Filed: Feb. 2, 2022
(86) PCT No.: PCT/GB2022/050274

§ 371 Date: Jul. 25, 2023

(30) Foreign Application Priority Data
Feb. 3, 2021 (GB) 2101514.4

(51) Int. Cl. G01N 27/407; H01M 8/0444; H01M 8/04664; H01M 8/04955

(52) U.S. Cl. CPC G01N 27/4071; H01M 8/04447; H01M 8/04671; H01M 8/04955

(57) ABSTRACT

The present disclosure provides a fuel cell comprising a first sensor comprising a membrane electrode assembly (MEA) comprising a plurality of electrodes 102, 104 and a membrane electrolyte layer disposed between the plurality of electrodes 102, 104, wherein the MEA is disposed between a first substrate 122 and a second substrate 120, wherein the first substrate 122 has at least one opening 112 to provide a gas flow path therethrough to one of the electrodes 104. The fuel cell also comprises an electrical control unit 402 to determine an electrical characteristic of the MEA, wherein the electrical characteristic is indicative of the gas composition of the gas at the one of the electrodes 104 and wherein the electrical control unit 402 will generate an output based on or in response to a change in the electrical characteristic.

The present disclosure relates to sensors and sensor devices, fuel cells comprising sensor devices, use of said fuel cells, use of said sensors, use of membrane electrode assemblies (MEAs) as sensors, sensor systems and methods of detecting gas or detecting a change in gas composition.

The present invention has particular application to fuel cells, for example solid-polymer-electrolyte fuel cells.

### BACKGROUND

Electrochemical sensors are commonplace, and their structure is typically a physical sensor which senses a gas by detecting a change in the conductivity of air in the presence of said gas.

Traditional electrochemical sensors rely on the oxidation of some solid species (for example lead or tin dioxide) to determine the presence of gas. This inherently limits the number of times a sensor can be used, as the presence of gas to be detected is consuming the sensor's fuel, and these type of prior art sensors can rapidly deteriorate and become unusable. Traditional electrochemical sensors are typically heated in order to account for environmental factors such as relative humidity. This is achieved by constantly heating the sensing element to high temperatures (>300° C.). This means such sensors act as a continuous and large parasitic power drain on any system they are part of. Such sensors need 2.25 W of power for around ten seconds on start up, and then 0.9-1 W of power during regular operation.

Traditional electrochemical sensors are often heated sensors which can take many hours to power up and reach the correct temperature. See for example the hydrogen sensors Hydrogen Click™ supplied by Mikroe, which rely on a sensor layer of tin dioxide which changes conductivity when hydrogen is present. This sensor needs 24 hours to heat up and reach the right temperature. This is typical of the sensors presently available in the art.

A fuel cell (e.g. a solid-polymer-electrolyte fuel cell) is an electrochemical device which generates electrical energy and heat from a reactant or oxidant (e.g. pure oxygen or air) and a fuel (e.g. hydrogen or a hydrogen-containing mixture, or a hydrocarbon or hydrocarbon derivative). Fuel cell technology finds application in stationary and mobile applications, such as power stations, vehicles and laptop computers.

According to British Standard 62282-2 for fuel cells, gas consuming fuel cells must have a sensor to monitor for internal gas leakage. Gas leakage sensors are an important part of ensuring the safety of fuel cells. Thus, there is a need for low cost, energy efficient gas sensors in fuel cells.

Existing sensors are relatively expensive, due to a high number of components and the use of expensive components. Existing sensors act as relatively large continuous parasitic elements and drain power generated by fuel cells as they need constant power taken from the fuel cells power output in order to operate. For the operation of fuel cells, especially those of a lower wattage such as fuel cells capable of producing less than 200 W, and in particular around 20 W, the loss of, for example, 1 W to 2 W of the power output could represent 5% to 10% of the power generated by the whole system. Currently available gas sensors act as a relatively large parasitic on fuel cells, especially the smaller scale fuel cells.

Sensors which work immediately on power up would be advantageous. It would also be advantageous to have sensors that are useable after multiple detection events, i.e. they do not rapidly deteriorate with detection events as the fuel of the sensor is consumed.

In view of the foregoing, it is desirable to provide an improved sensor device for sensing the presence, level of or leakage of gases, for example for use in fuel cells.

### SUMMARY OF THE INVENTION

According to a first aspect of the present invention, there is provided a fuel cell comprising a first sensor comprising a membrane electrode assembly (MEA) comprising a plurality of electrodes and a membrane electrolyte layer disposed between the plurality of electrodes, wherein the MEA is disposed between a first substrate and a second substrate, wherein the first substrate has at least one opening to provide a gas flow path therethrough to one of the electrodes. The fuel cell also comprises an electrical control unit to determine an electrical characteristic of the MEA, wherein the electrical characteristic is indicative of the gas composition of the gas at the one of the electrodes and wherein the electrical control unit will generate an output based on or in response to a change in the electrical characteristic.

The following discussion of the sensor while noted to be in a fuel cell in the first aspect of the invention apply mutatis mutandis to all aspects of the invention, particularly the second to the fifth aspects of the invention which follow. ‘Sensor’ and ‘sensor devices’ are used interchangeable throughout. All characteristics, components, compositions of and properties of the sensors described with relation to the first aspect of the invention can be applied to the other aspects of the invention.

A single pair of electrodes separated by an electrolyte membrane is a membrane electrode assembly (MEA). Each MEA comprises an electrolyte membrane, which is an ion-permeable membrane, e.g. a proton exchange membrane. An MEA also comprises two electrodes on either side of the electrolyte membrane. The electrolyte membrane allows ions (e.g. hydrogen ions/protons) but not free electrons, to pass through from one electrode to the other. The electrodes are electrically insulated from each other by the electrolyte membrane. When a ‘fuel’ (i.e. something the electrode will react with) is present adjacent to one of the electrodes a reaction occurs at this electrode, generating electrons and ions. The ions move between the electrodes across the electrolyte membrane and are consumed (react) at the opposing electrode. A circuit can then be formed between the two electrodes for the electrons to flow through, to generate an electrical current. Catalyst layers or integration of catalysts with the electrodes can accelerate reactions at both of the electrodes. MEAs can stacked up and utilised in fuel cells to generate electrical energy.

The inventors have unexpectedly discovered that MEAs can be used as part of relatively simple and low-cost gas sensors, which have particularly useful applications in fuel cells. This use and the sensor devices described herein are not reported anywhere else. The sensors can be used as ambient sensors and as an alarm or part of an alarm system to indicate that there has been a change in a gas composition in an environment where it would be desirable to detect a change in gas composition. The sensors described herein do not utilise a specifically trapped, enclosed or supplied ‘reference gas’, like those of the prior art. The ambient air or whatever gases the sensor it usually exposed to act as an equivalent to the “reference gases” of prior art sensors. Preferably, a reference (gas) for the sensors is ambient air, or the gas composition the sensor is usually exposed to. In a fuel cell the gas composition the sensor is usually exposed to will be ambient air. However, when used as a sensor outside of a fuel cell, the gas composition the sensor is usually exposed to could be different to ambient air, or it could be a total lack of any gas (i.e. a vacuum).

The first substrate of the sensor has at least one opening to provide a gas flow path therethrough (e.g. from an external surface of the first substrate, or from the ambient atmosphere surrounding the substrate) to one of the electrodes of the MEA. This means that the gas composition at the electrodes adjacent substrates with openings is the same or very similar to the gas composition at said substrate. Electrodes will react with the gas present at their surfaces. If there is a change in the gas composition at the surfaces of the electrodes then there will be a change in the reaction, or new reactions will take place. There will thus be a change in the electrical characteristics of the MEA upon a change in the gas composition at one or more of the electrodes of an MEA. A change in an electrical characteristic of the MEA indicates a change in the gas composition around at least one of the electrodes, or the change at respective substrate layers.

Preferably, the MEA comprises at least two electrodes and at least one of these electrodes on each side of the MEA are exposed to the atmosphere. Having both a sensing and a reference electrode exposed to the atmosphere ensures that standard atmospheric gas can be used as reference gas.

The sensor device is effectively a low power fuel cell, where an electrical characteristic will be detectable or measurable when a gas differential is present across the two faces, for example when hydrogen is present in a higher concentration on one face of the sensor (and thus one of the electrodes) than the other. The change in electrical characteristic can be determined to indicate the presence of a gas previously not present, or a change in the gas composition around the sensor.

An electrical control unit can determine an electrical characteristic of the MEA indicative of the gas composition of the gas at one of the electrodes and can then generate an output based on or in response to the change of electrical characteristic. This can generate a signal which could be generated to indicate a change in gas concentration around the sensor, i.e. the presence of a gas such a hydrogen. The signal can then result in a further action or event. The output or signal could be sent to some form of control module to affect a change in the wider system the sensor device is a part of. In a fuel cell, this could result in the fuel cell being turned off because the presence of a fuel gas in at the sensor would indicate a potentially dangerous gas leak and/or a fuel wasting gas leak. A control module itself (comprising a device to detect a change in the potential difference across the electrodes) may itself be monitoring for a change in the electrical characteristics of the MEA of the sensor devices described herein. The fuel cell or external control device connected to or controlling the fuel cell may comprise a control module. The electrical characteristic to be measured could for example be potential difference, open circuit voltage between the anode side and the cathode side of the MEA, current generated, capacitance or conductivity. More than one electrical characteristic could be determined at any one time or in any one device.

The proposed solution does not require a constant current flow in the sensor device to work, thus there is a reduction in parasitic power on any system utilising such a sensor when compared to the fuel sensors of the prior art. This is especially useful on smaller scale systems, for example fuel cells. The only power required from the proposed solution is from a device to measure any electrical characteristics or changes in electrical characteristics, i.e. no large parasitic load is associated with the sensor itself, as required in the traditional sensors which require a large amount of power to heat the sensors. The sensor devices described herein only require around 1-10 mW of power to operate. This is drastically less than the 0.9-1 W of power during regular operation of the prior art sensors described in the background section.

The sensor is smaller than prior art sensors, thus taking up less space in a fuel cell that could be used for other components. Again, this is especially useful in smaller fuel cells. These sensors are also advantageous over the described prior art sensors because they are ready for use faster than the described prior art sensors, which take many hours to power up and equilibrate to reach the correct temperature. The sensor devices described herein are in effect “always on” and have no warm-up time. Metal oxides in prior art sensors deteriorate at a much greater rate, rendering them only viable for a few uses. These sensors of the present invention can go through many more sensing events and uses than those of the prior art, which may deteriorate after only a few uses. The sensors described herein may go through tens of thousands of uses, due to the robust nature of the MEAs utilised. These sensors are more cost effective than the prior art sensors, in part because they use less components and thus are simpler to manufacture.

The presently described sensors are more sensitive than those of the prior art. These sensors are more sensitive than the prior art sensors, they can act at a nanovolt level as opposed to a millivolt level. A relatively minor change in gas composition is detectable by such sensors, because a very minor change in gas composition will inevitably lead to a change in the electrical characteristic(s) of the MEA. Because only a change in the electrical characteristic(s) need be detected, for example potential difference, this can result in a more sensitive measurement than the prior art sensors.

Safety of fuel cells is improved by having an accurate sensor present to detect for hydrogen leakage. Fuel cells utilising the herein described sensors are thus safer than those without. The improvements outlined above of using these sensors are especially beneficial when they are used in fuel cells.

Whilst the use of these sensors was initially imagined as being able to be placed in a fuel cell where leaked gases would be present, for example the top of a fuel cell, they could be utilised anywhere where a gas needs to be detected or sensed. The electrochemical components of the sensor device will change in order to be used for different gases to be detected.

Such sensor devices could be utilised in any environment where detection of gasses would be preferential.

Preferably, the first substrate and the second substrate are printed circuit boards (PCBs). Preferably, the sensor is formed by compression lamination of the PCBs.

The sensors described herein can tolerate more extreme environmental conditions than many sensors of the prior art due to the use of robust materials, for example, PCB materials, and the use of enclosed MEA structures which have a high tolerance to high temperatures and pressures. The sensors described herein are also able to withstand relatively high mechanical stresses.

Preferably, the sensor is mounted inside the fuel cell in a zone of the fuel cell where leaked gas would leak to such that any leak of reactant fuel from the fuel cell can be detected by the sensor.

Preferably, when a change in gas composition is detected by the sensor the fuel cell is turned off. Preferably there is a threshold electrical characteristic above or below which will result in the turning off of the fuel cell. The threshold value is set as part of the detection means. A threshold may need to be set so that a user or a system is not indicated of a change in gas concentration below said threshold. This could be to prevent incredibly minor differences in the gas composition, such as those differences which may occur in normal ambient air naturally (i.e. small variations in oxygen, carbon monoxide or carbon dioxide levels in the air). The threshold may be calculated by the electrical control unit to equate to a threshold gas level.

Preferably, the fuel cell further comprises means to indicate that an output based on or in response to the electrical characteristic has been generated.

Preferably, the second substrate has at least one opening so as to provide a gas flow path therethrough to one of the electrodes.

Preferably, at least one of the first substrate and the second substrate have multiple openings to provide multiple gas flow paths through that substrate. Preferably, just one substrate has multiple openings, the other just one opening.

Preferably, one of the first substrate or the second substrate has more openings than the other to provide more gas flow paths through that substrate to one of the electrodes than the other substrate, or wherein the opening or openings in one substrate are larger in dimension than the opening or openings in the other substrate to provide wider gas flow path(s) through that substrate to one of the electrodes. Preferably, one of the first substrate or the second substrate is dimensioned to have increate flow to one of the electrodes when compared to the other. Preferably, one electrode has more openings, channels or paths, or larger openings, channels or paths, whatever results in an increased gas flow to that electrode when compared to the other electrode. Preferably, there is more restriction of gas to one of the electrodes when compared to the access of gas to another electrode i.e., there is a reduced access to the electrode from the outside atmosphere, for example through less openings, channels or paths for gas to flow to the electrode.

Preferably, the MEA comprises at least two electrodes, wherein there is a gas flow path to each of the electrodes, and wherein one gas flow path is smaller or has a restricted gas flow compared to the other gas flow path.

These arrangements ensures that there will be a difference in gas volume, concentration and/or reaction at the two electrodes, creating a difference between the two electrodes. This is particularly advantageous in a fuel cell application, where having a sensor dimensioned to be able to sense a gas when the whole sensor is flood with gas, i.e. in the event of a gas leak, is particularly useful.

Preferably, the electrical characteristic is potential difference, open circuit voltage between the anode side and the cathode side of the MEA, current, capacitance or conductivity. More than one electrical characteristic can be measured at any one time.

Preferably, the electrodes and/or the sensor device further comprise a catalyst. This can facilitate the reaction of the electrodes with the gas to be sensed.

Preferably, the substrates further comprise a conductive metal layer, preferably wherein the conductive metal is copper. This can facilitate measurement of the electrical characteristics.

Preferably, wherein the fuel cell or the sensor comprises electronics capable of shorting the sensors to recalibrate the sensor.

Preferably, the electrodes are carbon paper electrodes comprising a carbon supported platinum catalyst.

Preferably, the electrolyte layer is a proton exchange membrane, preferably comprising a sulfonated tetrafluoroethylene electrolyte.

Preferably, at least one of the electrodes is an electrode which will react with the fuel of the fuel source consumed by the fuel cell.

According to a second aspect of the present invention, there is provided a sensor for detecting a change in gas composition. The sensor comprises a membrane electrode assembly (MEA) comprising a plurality of electrodes and a membrane electrolyte layer disposed between the plurality of electrodes. The sensor also comprises a first substrate and a second substrate, wherein the MEA is disposed between the first substrate and the second substrate. At least one of the first substrate and the second substrate has at least one opening to provide a gas flow path therethrough to one of the electrodes. When there is a change in the gas composition of the gas at the one of the electrodes an electrical characteristic indicative of the gas composition of the gas at the one of the electrodes can be determined to generate an output to indicate the change in gas composition detected by the sensor.

Preferably, one of the first substrate and the second substrate has more openings than the other to provide more gas flow paths through that substrate to one of the electrodes than the other substrate, or wherein the opening or openings in one substrate are larger in dimension than the opening or openings in the other substrate to provide wider gas flow path(s) through that substrate to one of the electrodes.

Preferably, the MEA comprises at least two electrodes, wherein there is a gas flow path to each of the electrodes, and wherein one gas flow path is smaller or has a restricted gas flow compared to the other gas flow path.

The sensor may have any or all of the other structural elements or properties of the sensors described in the fuel cell of the first aspect of the invention.

The sensor and sensor devices described herein can be used as a gas sensor in a fuel cell. Preferably, at least one of the electrodes is chosen or designed to be able to react with the fuel of the fuel source consumed by the fuel cell. Preferably, the sensor device is located inside the housing of the fuel cell. Preferably, the output will result in the turning off the normal operation of the fuel cell when a gas leak is detected.

According to a third aspect of the present invention, there is provided a sensor system comprising the sensor device as described herein as a gas sensor and an electrical control unit to determine an electrical characteristic of the MEA.

According to a fourth aspect of the present invention, there is provided use of a membrane electrode assembly (MEA) as a gas sensor. The MEAs as described herein can be used as sensors or sensor devices for gases.

According to a fifth aspect of the present invention, there is provided a method of detecting gas or detecting a change in gas composition, the method comprising determining a change in an electrical characteristic of an MEA, wherein the change in the electrical characteristic of an MEA indicates the presence or absence of a gas or wherein the change in the electrical characteristic of an MEA indicates a change in gas composition at the MEA.

Preferably the method of detecting gas or detecting a change in gas composition uses any one of the sensors described in relation to the first aspect of the invention, or any of the other aspects of the invention. Preferably, the method is part of a method of operating a fuel cell.

The sensors, sensor systems, uses of sensors and methods of detecting gas using the sensors described in the methods may have any of the other structural elements or properties of the sensors described in the fuel cell of the first aspect of the invention, all of these elements are interchangeable between aspects of the invention.

### BRIEF DESCRIPTION OF THE DRAWINGS

Embodiments of the present disclosure will now be described with reference to the accompanying drawings, by way of example only, in which:

FIG. 1 is a schematic diagram of an exploded embodiment of the invention;

FIG. 2 is a schematic diagram of an embodiment of the invention, where

FIG. 2A shows a top view, FIG. 2B shows a bottom or an alternative view of,

FIG. 2C shows another top (plan) view, FIG. 2D shows another bottom (plan) view, FIG. 2E shows a side view, and FIG. 2F shows an alternative side view of an embodiment of the invention;

FIG. 3 is a schematic diagram of a further embodiment of the invention, where FIG. 3A shows a top view and FIG. 3B another top (plan) view of the further embodiment of the invention;

FIG. 4 shows an exemplary sensor or system of the present invention, with the sensor connected to other components of a wider system;

FIG. 5 is a schematic diagram of a sensor of the present invention installed in a hydrogen fuel cell;

FIG. 6 shows a graph of voltage against time for a working embodiment of the invention demonstrating the use of a sensor of the invention as described herein;

FIG. 7 shows a graph of voltage against time for a working embodiment of the invention demonstrating the use of a sensor of the invention as described herein;

FIGS. 8a and 8b show two graphs of voltage against time for working embodiments of the invention, demonstrating the sensitivity using a sensor of the invention as described herein;

FIG. 9 shows a graph of voltage against time for a working embodiment of the invention demonstrating the repeated use of a sensor of the invention as described herein;

FIG. 10a shows a graph of voltage against time for a working embodiment of the invention demonstrating the use of a sensor of the invention as described herein under different environmental conditions, FIG. 10b shows voltage readings for the three different segments (A, B and C) of the graph from FIG. 10a;

FIG. 11 shows a graph of voltage against time for a working embodiment of the invention demonstrating the stability during use of a sensor of the invention as described herein; and

FIG. 12 shows a graph with the results from a further experiment utilising an oxygen sensor as described herein.

Like reference signs refer to like features throughout the drawings and the disclosure. Positional descriptions of sensors, e.g. “top”, “bottom”, “side”, are only relative terms used to describe specific embodiments and not meant to be limiting on the invention.

### DETAIL DESCRIPTION

Embodiments will now be described in detail with reference to the accompanying drawings. In the following detailed description numerous specific details are set forth by way of examples in order to provide a thorough understanding of the relevant teachings. However, it will be apparent to one of ordinary skill in the art that the present teachings may be practiced without these specific details.

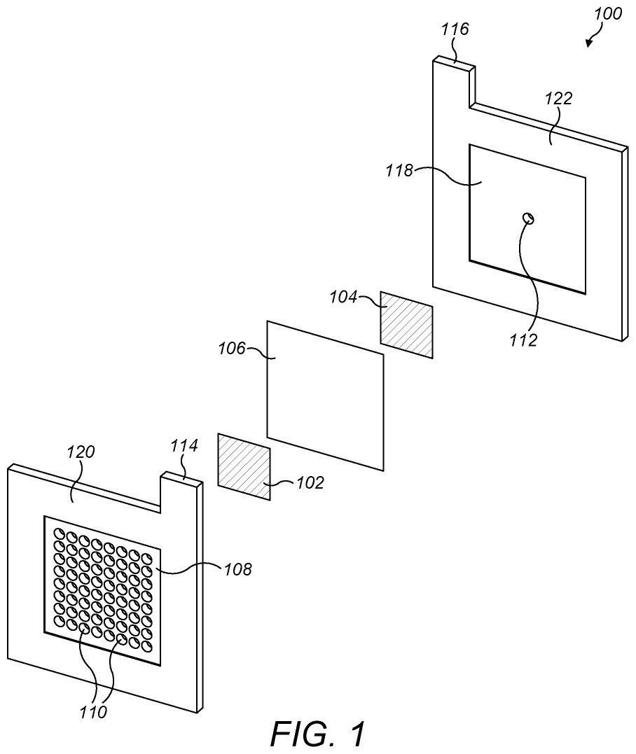

FIG. 1 is a schematic diagram of an exploded embodiment of a sensor device 100 of the invention, with substrate layers 120 and 122 shown outermost. An MEA comprising an electrolyte layer 106 sandwiched between two electrodes 102, 104 is arranged between the substrate layers 120, 122.

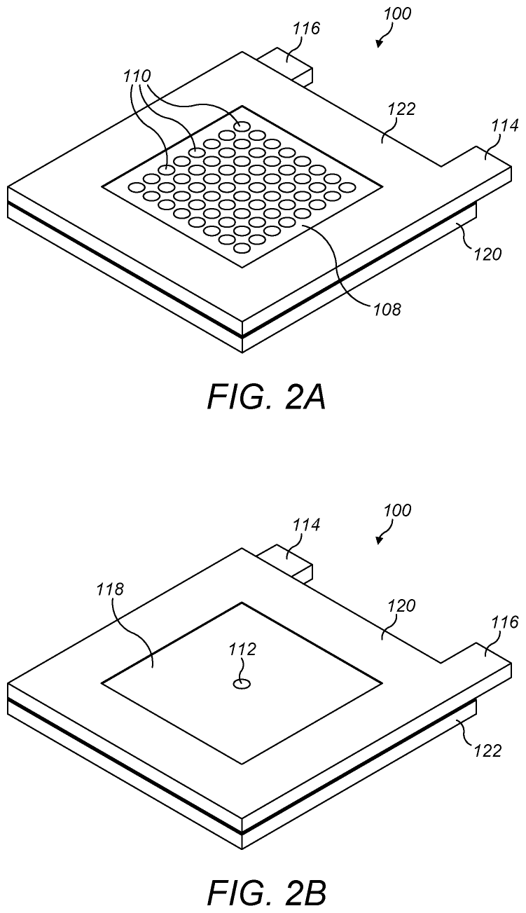

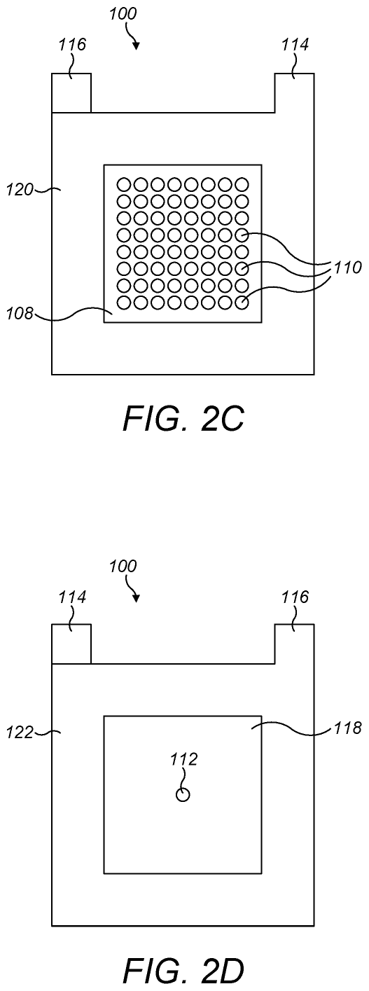

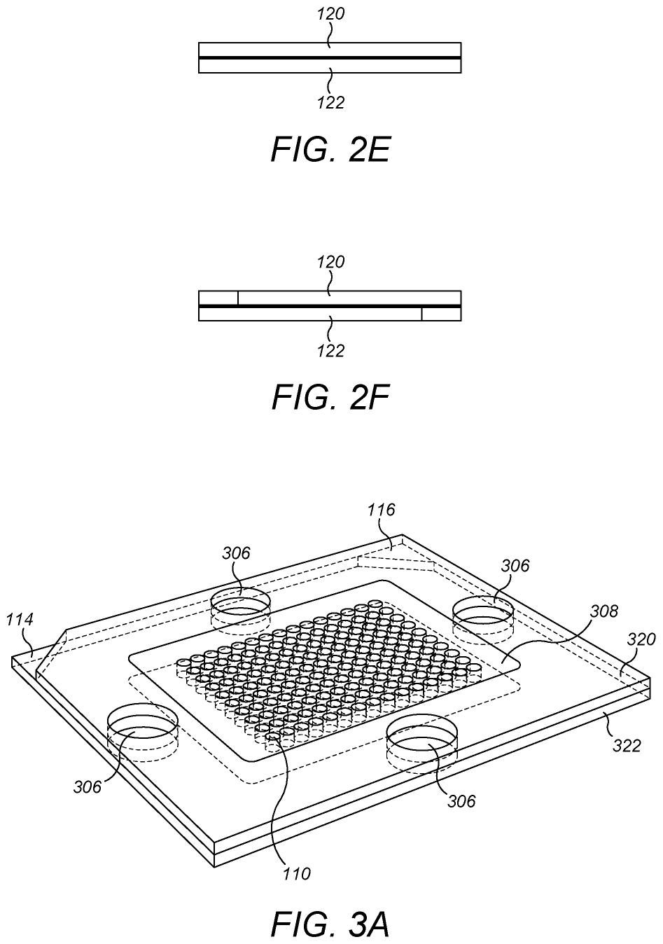

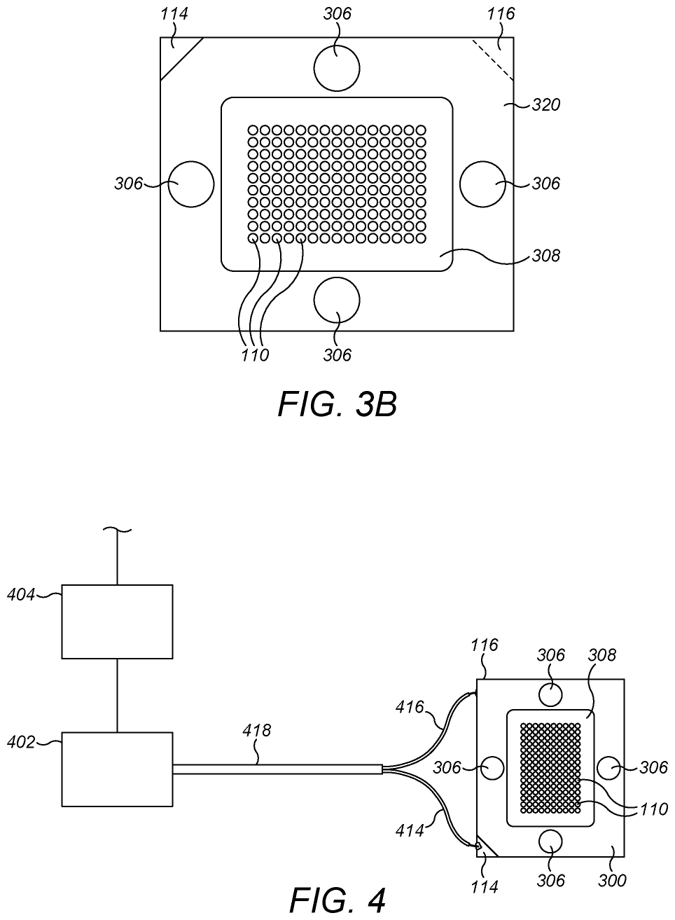

Electrodes 102, 104 may be referred to as ‘electrode layers’. Electrodes 102, 104 comprise catalysts. The electrodes 102, 104 are positioned in the centre of the sensor device 100, either side of the electrolyte layer 106, although the claims are not limited in this respect. These electrodes 102, 104 could be considered a ‘top’ and a ‘bottom’ electrode when the sensor device 100 is held horizontal (as shown in FIGS. 2 and 3). If the sensor device 100 was held vertically (as shown in FIG. 1) they could be considered ‘left’ and ‘right’ electrodes. Substrate layers 120, 122 could also be considered ‘top’ and ‘bottom’ or ‘left’ and ‘right’.

In the sensor device 100 the two electrodes 102, 104 are in contact with the substrate layers 120, 122, one electrode 102, 104 in contact with one substrate layer 120, 122. Substrate layers 120, 122 are in contact with the electrodes 102, 104 in order to allow the electrical characteristics of the device 100 to be measured. Substrate 122 can be referred to as a reference layer, reference voltage plate or reference substrate. Substrate 120 can be referred to as a sensing layer, or a sensing voltage plate, or a sensing substrate.

Point 116 is a reference plate measurement point or connection point and point 114 the sensing plate measurement point or connection point. An electrical probe can be connected in electrical communication with these points 114, 116 to detect or measure electrical characteristics of the MEA.

Through-holes or openings 110 and 112 run through both substrate layers 120, 122. These through-holes provide flow paths or channels through the substrates 120, 122, between each of the electrodes 102, 104 and the gas composition or atmosphere local to and surrounding each substrate layer 120, 122. Thus, the through-holes 110, 112 in substrates 120, 122 provide a flow path from the outside the sensor device 100 to the electrodes 102, 104 inside the sensor device 100, allow the gas composition around the sensor device 100 to diffuse to the electrodes 102, 104 of the MEA to the electrodes 102, 104 of the MEA. Through-holes 110 through substrate layer 120 provide a flow path from the gas composition around substrate layer 120 to electrode 102.

In the embodiment shown in FIGS. 1 and 2 substrate layer 122 only has a single through-hole 112 provides a flow path from the gas composition around substrate layer 122 to the other electrode 104. A single through-hole provides a more restricted flow path access or reduced possible flow of gas to electrode 104, when compared to electrode 102 which has multiple flow paths to it due to multiple through holes 110 through substrate 120. As described herein, restricting the access of gas to one of the electrodes when compared to the access of gas to another electrode results in a difference in gas diffusion properties and hence reaction rate between the two electrodes, producing a voltage difference.

‘Through-Holes’ could be referred to or defined as openings, holes, channels, passages, flow paths, paths or other such terms in the art for means which would allow diffusion of gas to an electrode. Whilst FIG. 1 shows multiple through-holes 110 in substrate 120 and a single hole 112 in substrate 122, the size, shape and number of through-holes can be varied in the sensor devices and systems described herein. These can be tailored to provide control over the reaction of gases at the electrodes. As demonstrated in FIG. 1, there could be a single through-hole 112 in the substrate 122 to provide access to the electrode 104 or there could be multiple through-holes 110 in the substrate 110 to provide access to the electrode 102. A hole could be the size of the entire electrode area, providing a completely open-faced electrode as part of an MEA held by a substrate. The through-holes can be shaped in any way, for example circular, square, rectangular or hexagonal through-holes. The through-holes can be patterned across the substrate however best to suit the design of the sensor. As noted elsewhere, through-holes may only necessarily be present on one substrate of the sensor devices described herein.

The flow paths created by the through-holes 110, 112 through substrates 102, 104 between the electrodes 102, 104 of the MEA and the gas composition surrounding each substrate allows the diffusion of gas to the electrodes 102, 104 of the MEA. Thus, a change in the gas composition around at least one of the respective substrate layers 120, 122 will result in a change in the gas composition at the electrodes 102, 104 themselves.

No through-holes anywhere in the sensor device 100 of FIG. 1, or any of the embodiments of the invention described herein, run through the MEA itself (so not through the electrodes 102, 104 or electrolyte layer 106).

In the exemplary embodiment depicted in FIG. 1, the substrates 120, 122 are printed circuit boards (PCBs) and sensor device 100 is a laminated structure although the claims are not limited in this respect. Lamination results in a solid structure, with contact between the individual layers, providing a monolithic, lightweight, and sealed sensor device 100. A copper layer is on both sides of each substrate plate 120, 122, with a non-copper square 108, 118 etched away in the centre of each substrates 120, 122. The particular advantages of PCB technology are described below.

Schematics of a sensor device 100 according to the present invention are shown in FIGS. 2A-2F. Like numbering will be used to describe like features depicted in FIG. 1.

An angled plan view of one side of sensor device 100 is shown in FIG. 2A (sensing side), a second angled plan view of the other side of sensor device 100 is shown in FIG. 2B (reference side), a plan view of one side of sensor device 100 is shown in FIG. 2C (sensing side), a second plan view of the other side of sensor device 100 is shown in FIG. 2D (reference side), a side view of the sensor device 100 is shown in FIG. 2E and an alternative side view of the sensor device 100 is shown in FIG. 2F.

In FIGS. 2A, 2B, 2C, 2D, 2E and 2F, laminated sensor device 100 has two printed circuit board (PCB) layers 120 and 122. The particular advantages of PCB technology are described below. The MEA shown and described in FIG. 1 is not visible as the device is not shown exploded, but is present in the device 100 of FIG. 2. FIGS. 2A and 2C show through-holes 110 through substrate 120 and FIGS. 2B and 2D show through-hole 112 through substrate 122. A copper layer is provided on both sides of each substrate plate 120, 122, with a non-copper squares 108, 118 etched away in the centres of each substrates 120, 122.

The diameter of the through-holes 110 and 112 are 1 mm. The length and width of the sensor device 100 including connection points 114 and 116 is 23 mm and is 20 mm not including connection points 114 and 116. The distance from one edge to the through-hole 112 of substrate 122 is 10 mm. The distance from the edge to the closest through-holes 110 of substrate 120 are 5.5 mm. The total thickness of the sensor device 100 is 2 mm. Each substrate layer 120, 122 is 1 mm thick. Connection points 114 and 116 are present on substrate layers 120, 122, as described in reference to FIG. 1. The claims are not limited in any of these respects.

Sensor device 100 of FIGS. 1 and 2 has 1 cm 2 MEA made of a sulfonated tetrafluoroethylene electrolyte layer (10 μm thick) and two gas diffusion electrodes made of carbon paper with a carbon supported platinum catalyst (200 μm thick, when uncompressed). The sensor has a 1 mm thick sheet of PCB (FR4 material) either side of the MEA with a 18 μm layer of copper either side of the PCB layer. Any copper layers exposed to the MEA are coated in a 20 μm thick layer of carbon ink.

Sensor device 100 can be connected to an electrical sensor unit or means to detect or measure an electrical characteristic of the MEA. For example, the electrical sensor unit can be used to detect or measure the potential difference across the electrodes of the MEA, or the open circuit voltage between the anode side and the cathode side of the MEA. Electrical connections can be for example in the corners of each substrate layer 120, 122, at connection points 114 and 116. Lamination of the device 100 results in contact between the individual layers. Because of the copper layer etched onto the substrates 120, 122 and the laminated structure of the device 100 provides an electrical connection in each corner of each substrate layer means that the electrical characteristics of the MEA/substrates/electrodes 120,122/102,104 can be determined, sensed detected or measured. Plated through-holes or vias could also be introduced into the sensor device 100 in order to be used to measure the electrical characteristics on the outer surfaces of the device. Plated through holes could provide a low-resistance electrical pathway from one surface (i.e. the copper face in contact with the electrode) to another (i.e. the outer face of the device) to make measurement of voltage more convenient.

The electrical control unit to determine an electrical characteristic of the MEA can be a potential difference or voltage measuring apparatus or module of any appropriate known type in the art. Monitoring of the potential difference across the electrodes of the sensor allows for the sensing of any change in gas composition or the presence of a gas to be detected at the sensor device 100. A module could be, for example, an ADC (analog to digital converter) on a microcontroller. An ADC is used to convert an analogue signal such as voltage to a digital form so that it can be read and processed by a microcontroller. A microcontroller could have a built-in ADC converter, or an external ADC converter can be connected to any type of microcontroller. An ADC has very low or no parasitic on the system as a whole while the system is doing something else.

In operation, when not indicating a change in a gas composition or the presence of a gas., i.e. when the sensor device 100 is in the standard atmosphere for such a sensor device 100, for example ambient air, sensor device 100 will normally be exposed to the normal or standard atmosphere around the sensor device 100. Electrical characteristics can be determined for normal operation. For example, if the electrical characteristic to be determined is potential difference, this will provide a baseline or a “normal” potential difference across the electrodes 102, 104. This potential difference will depend on the nature of the electrodes 102, 104 and whether or to what level they electrodes 102, 104 normally react with any gases present in the atmosphere. One or both of the electrodes 102, 104 may constantly be reacting with gases present in normal atmosphere, e.g. reacting with oxygen. There will thus be a resting or normal potential difference across the electrodes 102, 104.

If there is a change in the gas composition around the sensor device 100, i.e. if there is an increased concentration of a gas such as hydrogen, carbon monoxide or oxygen, or a new gas present which was not previously present, due to the through holes 110, 112, the gas composition at one or more of the electrodes 102, 104 will also change. If the MEA electrodes 102, 104 are of a material composition which will react with that newly present gas, then a reaction will take place at one or more of the electrodes 102, 104 and the electrical characteristics of the MEA will change, e.g. the potential difference across the two electrodes 102, 104 will change. The electrical characteristics will change due to the transfer of the generated species from the reaction.

In normal operation, it is likely that just one side of the sensor device 100 will be exposed to a significant change in gas composition. Both sides may be exposed to some gas, but due to positioning of the sensor device 100, or the layout/size/design of the through-holes through the substrates, one side likely exposed to a greater amount of gas. This will result in a change of the electrical characteristics of the MEA between the two electrodes 102, 104 or a change in the potential difference that already exists between the two electrodes 102, 104. The reference electrode 104 opposite to the sensing electrode 102 can react with oxygen in the air to consume the ions generated from the sensing electrode 102 which pass through the electrolyte membrane. When the sensor device 100 is in ambient air, there will be consumption of oxygen in the air.

The sensors described herein can have a side specifically intended to be exposed to the gas to be sensed, i.e. positioned so that just one side of the sensor would encounter the gas to be sensed. Thus, the sensor can be directional—having a gas sensing side and a non-gas sensing or reference side. This non-gas sensing or reference side may just be exposed to ambient air, e.g. outside of a device which the sensor is placed, with the sensing side positioned inside a device. For example, when the sensor electrochemistry is designed to react with hydrogen, the reference side could be positioned to not be exposed to hydrogen, or a reduced level of the hydrogen compared to another side. Then, when hydrogen (or an increased hydrogen presence) is present on the sensing side of such a sensor, it will react with the electrode, which will act as an anode, whilst the other electrode will consume oxygen from ambient air, acting as a cathode. In some embodiments described herein, the sensors may not utilise a specifically trapped, enclosed or supplied ‘reference gas’, like those of the prior art. In those, the ambient air or whatever gases the sensor it usually exposed to act as an equivalent to the “reference gases” of prior art sensors.

In the exemplary embodiment shown in FIGS. 1 and 2, upon a change in gas concentration at the substrate 120 a change in the potential difference between the two electrodes 102, 104 would be detectable across the measurement points 114, 116 on both substrates 120, 122. If both substrates 120, 122 in sensor device 100 were exposed to gas equally at the same time, due to the different flow paths resulting from the different through-holes 110 and 112 in substrates 120 and 122, there would still be a potential difference across the electrodes 102, 104, despite them being the same electrode types, because they would be exposed to a different account of gas due to the difference in flow paths to the electrodes 102, 104.

Thus, when there is a change in the gas composition it is possible to detect a change in the potential difference across the electrodes. This can be detected with a means to detect the potential difference across the two electrodes as described above. Such means can be included in the sensor or as part of a wider sensor system or apparatus the sensor is a part of.

In addition to, or alternatively to, detecting a change in the potential difference across the electrodes, the sensor may detect a current generated by the electrodes upon reaction with a gas. When a reaction takes place at an electrode electrons are generated and a load can be applied so that the flow these can be channelled (e.g. down wires), generating a current. The sensing and reference sides could be arranged to have a low resistance set up and an in-line ammeter could measure the current. Any changes in current would be indicative of a change in concentration of gas. Current could be detected or measured as another indicator or used to power a means to indicate that there has been a change in the gas composition around the sensor. Means to detect the generation of a current can be included in the sensor or as part of the wider sensor system or apparatus the sensor is a part of.

Detecting a change in electrical characteristics can in turn result in an output. This output could be a signal generated in order to signal a change in gas concentration around the sensor, i.e. the presence of a gas such a hydrogen or increased concentration of a gas such as hydrogen. This output can then result in a further action or event. A signal could be sent to a means to indicate that there has been a change in the gas composition or the detection of a gas at the sensor (e.g. an alarm or a warning light to indicate to a user there is a change in gas concentration e.g. a gas leak), or a signal could be sent to some form of control module to affect a change in the wider system the sensor device is a part of. In a fuel cell, this could result in the fuel cell being turned off (possibly via a control module) because the presence of a fuel gas in at the sensor device would indicate a gas leak. A control module (comprising a means to measure a change in the potential difference across the electrodes) may monitor, measured or detect the change in electrical characteristics of the MEAs of the sensor devices described herein, and enact change in a system the sensor is part of, e.g. a fuel cell.

Schematics of a further electrochemical sensor device according to the present invention are shown in FIG. 3. An angled plan view of sensor device 300 is shown in FIG. 3A and a further plan view of sensor device 300 is shown in FIG. 3B.

In FIGS. 3A and 3B sensor device 300 is shown with two printed circuit board (PCB) layers 320 and 322 visible. An MEA as described in reference to FIGS. 1 and 2 is provided/arranged between these two substrate layers 320, 322, but is not visible in FIG. 3.

In FIGS. 3A and 3B laminated sensor device 300 has two printed circuit board (PCB) layers 320 and 322, which can act as a reference and sensing substrates, as described with respect to FIGS. 1 and 2. In the embodiment of the invention depicted in FIG. 3 the through-holes 110 through the substrate layers 320, 322 are shown as equal in number and dimension through both substrate layers 320, 322. Points 116 and 114 are the measurement points or connection points. At these points 114, 116, the potential difference across the electrodes can be detected or measured by providing an electrical detection unit in electrical communication therewith to detect the potential difference. A copper layer is provided on the outside of each substrate plate 320, 322, with a non-copper square 308 etched away in the centre of each substrates 320, 322.

The total thickness of sensor device 300 is 2 mm. Length of the sensor device 300 is 40 mm, width is 33.11 mm and the diameter of hole 306 is 5.2 mm. Each PCB substrate layer 320, 322 is made of FR4 material and is 1 mm thick, with a 18 μm layer of copper either side of the PCB substrate layer. Any copper layers exposed to the MEA are coated in a 20 μm thick layer of carbon ink. Sensor device 300 has a 5 cm 2 MEA comprising a sulfonated tetrafluoroethylene electrolyte layer (10 μm thick) and two gas diffusion electrodes made of carbon paper with a carbon supported platinum catalyst (200 μm thick, when uncompressed).

Mounting holes 306 are also provided through both substrates 320, 322 and the electrolyte layer. These are not in contact with the electrodes and do not go through the electrodes, and thus do not provide any flow path from the gas composition surrounding each respective substrate layer 320, 322 to the electrodes. These can be used to mount the sensor device 300.

FIG. 4 shows an exemplary sensor device 300 of FIGS. 3A and 3B connected to parts of a wider system. Connection points 114 and 116 are shown in electrical communication with conducting means, 416 and 414, which may for example comprise electrical cables or wires.

Wires 416 and 414 connect sensor device 300, via connection wire binding/sheath 418, to electrical control unit 402, here a potential difference detection means 402. Wires 414, 416 could be a two core wire/cable, or alternatively bound together with a wire sheath 418 as shown. Potential difference detection means 402 is connected to control module 404. Control module 404 is shown with connection to the rest of a larger system. In further embodiments, potential difference detection means 402 and control module 404 could be part of the same single module or device. Wires can be attached with solder but can be attached using surface mounted electrical connectors, clip connectors, or detection means 402 could be connected to a sensor device 300 by a direct metal/metal interface (e.g. a push fit connector), or they could be nut and bolted together.

Control modules 404 may provide means to shut off the fuel cell, on sensing a gas or a change in gas composition that indicates a leak inside the fuel cell. Control modules 404 may be controlling other aspects of wider devices that the sensors described herein are a part of.

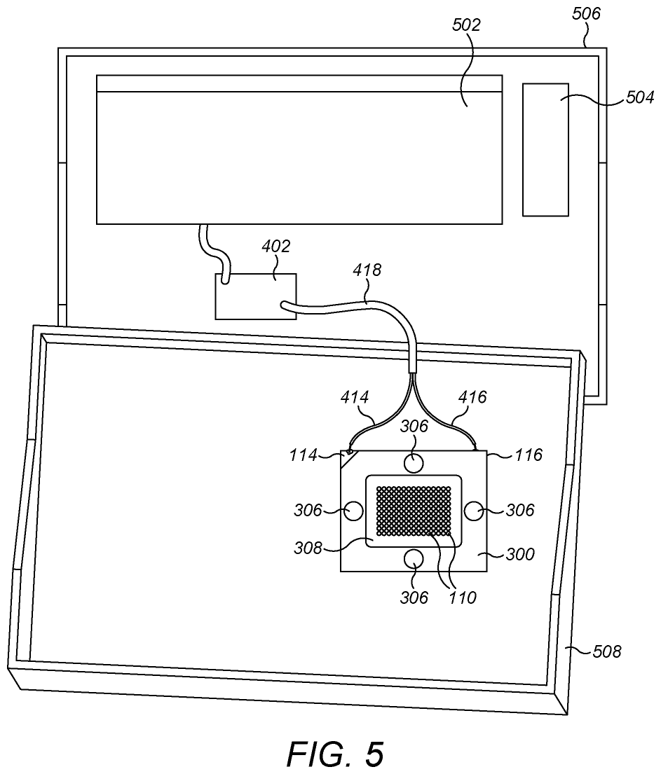

FIG. 5 shows an exemplary sensor device 300 of FIG. 4 installed in a simplified schematic of a hydrogen fuel cell 500. Parts of the fuel cell are not shown to scale. Connection points 114 and 116 are shown connected to wires 416 and 414 shown. Wires 416 and 414 connect sensor device 300, via connection wire binding 418, to potential difference detection means 402. A wire connecting potential difference detection means 402 to the further parts of the fuel cell is shown in FIG. 5. 504 is shown which will cool fuel cell stack 502. Lower casing 506 is shown, with fuel cell casing lid 508 shown holding the sensor device 300.

One side of sensor device 300 (the side of substrate 320) is directed down towards the inner workings of the fuel cell. The positioning of the sensor device 100 in this way means that when gas leaks it will rise inside the fuel cell and come into contact with the sensor. Due to through-holes 110 in substrate 320, hydrogen gas will diffuse to the electrode on that side of the MEA via the through-holes. In a fuel cell it is likely this gas will be pressurised and thus gas will contact the sensor almost immediately (i.e. in under 1 second's time) when any gas leaks. The gas will react at the electrode, causing a change in potential difference across the electrodes. This change can be detected or measured by a potential difference detection means 402. This can then result in a signal to generate an alarm and/or shut down operation of the fuel cell, as part of a wider safety system.

For all embodiments of the invention described herein, the electrochemistry of the MEA can be varied, i.e. changing the electrodes, catalysts and electrolytes, so that sensors to different gases or sensors with different levels of sensitivity can be produced. Electrodes of different compositions can react with different gases. Electrolyte material and suitability can also be varied to allow different ions to pass through, allowing sensing of different gases which produce different ions. In this manner, sensors can be tailored to detect different gases, based on the MEA electrochemistry.

For all embodiments of the invention described herein, the electrolyte layer can be any electrolyte membrane that allows ions (e.g. hydrogen ions) but not free electrons to pass through from one electrode to the other, for example, a sheet of Nafion™ membrane, a sulfonated tetrafluoroethylene electrolyte layer, a GORE-SELECT® Membrane (which is based on a ePTFE reinforced electrolyte), a Aquivion® ionomer or a copolymer of Tetrafluoroethylene (TFE) and Sulfonyl Fluoride Vinyl Ether. Electrolytes which allow proton exchange are suited for gases such as H₂ or H₂S.

For all embodiments of the invention described herein, the electrodes can be any electrodes which will react with the gas that the sensor is designed to detect. Such electrodes are well known to those of skill in the art. Both electrodes can be made of the same material. The electrodes can comprise the same or different materials.

Electrodes can be constructed to react with gases it would be desirable to detect, e.g. combustible gases. For example, Hz, CO, H₂S, CO₂ could be detected with the right electrodes and electrolyte combinations.

A single sensor could comprise a plurality of MEAs stacked or arranged accordingly. These could be all of the same type to detect the same gas, or a single MEA could comprise a plurality of different MEAs to react with different gases. A single sensor device could have multiple different electrodes and/or MEA structures of different types, designed to react with different gases, generating a detectable change upon the presence of different gas types in a single layer of a sensor device, or multiple electrode layers of a device. For example, 3 different sensing electrodes could be arranged counter to a single reference electrode, and the sensing electrodes or electrolytes adjacent to the electrodes could comprise different materials or comprise different catalysts designed to react with different gases or allow different ions to pass through, in order to be able to sensitively detect different gases in a single sensor device.

Electrodes can also comprise catalysts. A catalyst layer on the electrodes or a catalyst embedded in the composition of the electrodes accelerates a reaction with the gases to be detected. The catalyst will be a suitable catalytic material for the reactions of interest, as is commonly understood by a researcher skilled in the art of producing MEAs. For example, the catalyst layer may be composed of platinum nanoparticles deposited on carbon and bound with an proton conducting polymer (e.g. Nafion™), as described in “PEM Fuel Cell Electrocatalysts and Catalyst Layers Fundamentals and Applications”, Jiujun Zhang (Ed.), 1st Edition., 2008, XXII, 1 137 p. 489 illus., Springer-Verlag London, ISBN: 978-1-84800-935. Suitable catalyses would be known to those of skill in the art, for example palladium, iron, vanadium, aluminium, nickel based catalysts could be appropriate. For example, an iron catalysts supported on carbon electrodes would be suitable for CO₂ detection.

Because the sensors are potentially so sensitive, the detection of the change in potential difference, or the detection of the current generated by the MEA, may need to have a threshold value set as part of the detection means. A threshold may need to be set so that a user or a system is not indicated of a change in gas concentration below said threshold. This could be to prevent incredibly minor differences in the gas composition, such as those differences which may occur in normal ambient air naturally (i.e. small variations in oxygen, carbon monoxide or carbon dioxide levels in the air).

In use, separate electronics could be used to short the sensors regularly to aid in recalibrating the sensor to zero, by resetting both sides of the MEA to the same conditions. This could aid in improving detection and account for standard minor changes in atmosphere and gas composition. By shorting the sensors, effectively the electrode surfaces are returned to the same concentration. Following the release of the short any measured differences in potential can be attributed to new gas present at the sensor surface. Shorting could be carried out periodically, with set timings, to ensure a constant resetting of the sensor device to account for atmospheric variation. Shorting can also be automatically triggered or manually triggered after a detection event, to allow the sensors to continue to be used.

Multiple sensors device as described herein may be linked to one another as part of a wider sensor system comprising multiple sensors.

The ‘the atmosphere around the sensor’ or ‘atmosphere’ as used herein refers to the standard or normal atmosphere around the sensor. This could be different in different environments. This could be ambient atmosphere, i.e. the standard air composition where the sensor is being used, or this could be a total lack of gas around the sensor, i.e. it could be used in a vacuum (e.g. glovebox) environment. In that scenario, a change in gas composition could just be the mere presence of any gas. This may just result in a reaction and subsequent change in potential across a single electrode and a detection or sensing of gas.

The environment the sensor is set up in could affect performance, for example temperature, humidity, ambient atmosphere or standard atmosphere composition around the sensor composition. However, because the sensors are capable of detecting any change in the gaseous composition, the sensors, methods and uses described herein advantageously can be used in a variety of different environments. The fact change is always being compared to a base line or a reference allows these to be used in any environment. This offers advantages over sensors of the prior art, which may be more reliant on only working in certain environmental conditions, e.g. at an ideal operating temperature or humidity level. The sensors described herein can tolerate more extreme environmental conditions than many sensors of the prior art due to the use of robust PCB materials, and the use of enclosed MEA structures which have a high tolerance to high temperatures and pressures. The sensors described herein are also able to withstand relatively high mechanical stresses.

The substrates of the sensor devices, and other components of sensor devices where appropriate, can be constructed of Printed Circuit Boards (PCB). Individual layers can be constructed from PCBs which can be adhered together into a solid structure using an epoxy-containing glass fibre composite. The PCBs may be fabricated from pre-impregnated composite fibres, such that they contain an amount of the material used to bond the individual layers together and to bond the MEAs to the PCBs, or a pre-impregnated composite fibre mask may be applied to the PCBs. The MEAs may be laser bonded onto a PCB, thereby creating a layered structure. PCB boards can be laminated together. The gaps between the electrodes, and the sealing achieved in these gaps by the epoxy resin, prevents mixing between layers, i.e. stopping a gas diffusing from one substrate to another and affecting the potential difference measurement.

PCB technology has the advantage of enabling the elements to be manufactured in large quantities and at low cost. For example, multiple sensors can be manufactured at the same time, by using thin laminate boards which are stacked and then simultaneously routed. Individually routed boards are then stacked and laminated together. PCB structures also have a high mechanical strength and when laminated together provide a solid structure, with good contact between the individual layers. Accordingly, a monolithic, light, and completely sealed structure is produced. PCBs can be used as end boards if required. Both fuel cells and sensor devices for fuel cells can be constructed from PCBs. The construction of fuel cells from PCBs and their advantages are described in WO2013164639, which is incorporated herein by reference.

To create the sensor as described herein, the substrates and MEA are laminated together Lamination is achieved by heated compression of the boards. For example, this can be carried out at 180° C. for one hour. This gives sufficient time for the heat to pass through all the layers of the board. At this temperature, the epoxy or phenolic resin can cure, bonding adjacent layers together, ensuring good electrical contact and forming gas-tight seals.

For all embodiments of the invention described herein, a thin layer of copper can be applied to the outer side of each substrate layer and etched away (for example using a mask) to retain a desired conductive pattern. The copper layer can be applied by electroplating. The copper can be etched away to create non-copper rectangle in the centre of the sensor device, e.g. rectangles 108, 118 and 308 and in FIGS. 1, 2 and 3 as described above. The copper is etched away from the area which sits above and below the electrodes. This ensures that copper is not present in the active area of the MEA, so that copper does not react with the electrode when moisture is present, causing unnecessary electrode corrosion. The etched copper area can correspond to the housing that the senor will go in, for example in order to connect the sensor to voltage detection means, or to hold the sensor in place in a wider system.

A corrosion resistant coating that may be applied to the copper surfaces in the sensors. This coating is used to passivate the copper surface of the PCB and stop the copper layer from corroding. This is advantageous because the conditions in a fuel cell are very acidic and oxidizing, under which conditions copper corrodes. If the copper is oxidized from Cu⁰ to Cu²⁺, this could lead to the disintegration of the copper surface of the PCB, and the consequently, the sensor device. The coating must be stable and must not undergo any electrochemical or chemical degradation within the fuel cell environment. At the same time the material must be hard-wearing, as it must not become detached from the copper surface.

Sensor coatings may comprise carbon ink coatings. Carbon inks for coating copper of PCBs are known and commercially available. A carbon ink is a material comprising a carbon-based material and an organic binder. A carbon ink as described herein may preferably be a conductive carbon ink. A carbon ink as described herein is in line with the general understanding in the art. Generally, a carbon ink is an ink that may be easily screen printed and comprises carbon as a pigment along with a binder. A carbon ink may have a dynamic viscosity of between about 2.9 poise and about 7 poise, when calculated at 20° C. and 1 atmosphere. Therefore, a carbon ink may be a viscous material. Dynamic viscosity may be measured by a viscometer, calibrated to the viscosity of distilled water at 20° C. and 1 atmosphere (0.01 poise). When a carbon-based material is provided as a component in a carbon ink, the organic binder comprises the resins that are also used to form carbon inks. Exemplary carbon inks include PCB1 Sunchemical ink 2sp, PCB3 Sunchemical ink 2sp revisited and PCB4 Sunchemical ink (last) 2sp. carbon inks prevent corrosion of copper in the sensor devices. If copper were to corrode, it could poison the catalyst, reducing the accuracy of your measurement.

Sensor devices described herein are especially suitable for use in fuel cells. A fuel cell (e.g. a solid-polymer-electrolyte fuel cell) is an electrochemical device which generates electrical energy and heat from a reactant or oxidant (e.g. pure oxygen or air) and a fuel (e.g. hydrogen or a hydrogen-containing mixture, or a hydrocarbon or hydrocarbon derivative). Fuel cell technology finds application in stationary and mobile applications, such as power stations, vehicles and laptop computers. Fuel cells need a sensor to monitor for internal gas leakage. Thus, the sensor devices described herein can be placed inside the housing of a fuel cell and can be used to detect the presence of a gas.

A housing for a fuel cell assembly can have an electrochemical sensor device as described above mounted proximate to a leakage detection zone of the fuel cell such that any leakage of the reactant fuel from the fuel cell is detected by the electrochemical sensor. Sensors could be connected up to be able to result in the turn off normal fuel cell operations when detecting a leak of a gas. Sensors can be mounted directionally in fuel cells, so that the sensing side is directed to the likely direction of a gas leak. The fuel cells, fuel cell boards and components may be constructed from any suitable and desirable material, such as PCB, graphite or a metal.

Existing sensors act as a large parasitic on power generated by fuel cells, as they need a constant current in order to operate, as described in the background section. The sensor devices described herein do not act as a parasitic drain on fuel cell power because the sensor devices described herein do not require a constant current flow, there is a reduction in parasitic power and fuel cell systems can become more efficient whilst being able to sense gases.

Thus, the sensor devices described herein are especially useful on fuel cells which are only capable of generating a smaller amount of power (e.g. 20 W fuel cells), where having a large (by % of power output) parasitic drain by a known sensor of the prior art would be especially detrimental to the power output. A sensor requiring 1 or 2 W of power to function, due to always needing a current supply, would be a large % drain on a lower wattage fuel cells. For the operation of fuel cells, especially those of a lower wattage such as fuel cells capable of producing less than 200 W, and in particular around 20 W, the loss of 2 W of the power output could represent 10% of the power generated by the whole system.

Larger fuel cells i.e. those holding a larger gas (fuel) volume, may require a greater number of sensors to detect gas leakage in different part of a fuel cell, so the usefulness of the solution also scales with fuel cells which hold more gas volume.

As described above, the sensor devices could be used for example in an inert gas type environment (such as a ‘glove box’) in order to sense the presence of an unwanted, e.g. oxygen in an air leak.

The sensor devices described herein allow for a differential of pressure across the two surfaces. Thus, the sensor devices described herein can be used in pressurised environments. The sensor devices are suited for use in high pressure environments, unlike many prior art sensors. The structure of the device may be adjusted to account for a pressure differential across the device, for example increasing the thickness of the membrane, thicker membranes for higher pressure differentials. Such devices could be designed to be used as in line sensors, for example detection of hydrogen in gas lines, or detecting a particular contaminant(s) in a pressurised pipeline. One or both sides of the sensor can be pressurised. The sensors could be used in small gas lines, or large-scale gas lines.

In addition to as part of fuel cells or as in-line gas sensors, the sensor devices described herein could also be used in other scenarios where sensing the presence of a gas may be advantageous. For example, in manufacturing plants where gas leaks may be dangerous (e.g. a fuel call manufacture plant) or as part of gas sensing devices in homes (e.g. carbon monoxide sensors).

### Examples

A hydrogen sensor device as described herein (such as the exemplary embodiment shown in FIGS. 3, 4 and 5) was demonstrated to act as a sensor of hydrogen gas. One side of the sensor was exposed to hydrogen gas and the other side was used as a reference electrode, exposed to ambient air.

The sensor was mounted such that the reference electrode was exposed to air whilst not being vulnerable to hydrogen exposure. Hydrogen was briefly passed over the working electrode in such a manner that represents a significant hydrogen leak (mildly restricted flow, slightly pressurised). In this bench-top test, the voltage was recorded every 100 milliseconds using a potentiostat.

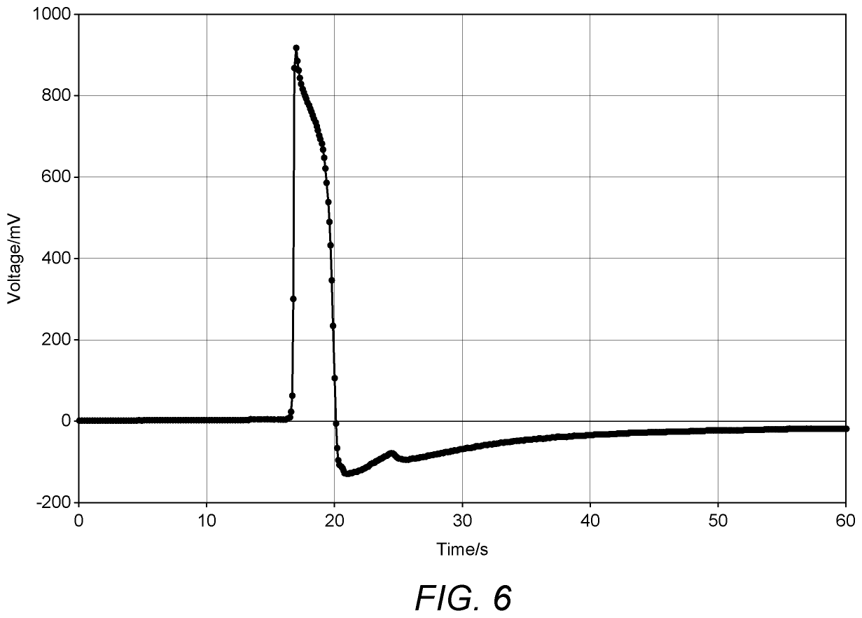

FIG. 6 shows a graph with the results from the experiment. A voltage trace of sensor over the course of a minute was recorded. The measuring side is exposed to hydrogen for 1 second at 16 seconds. Each circle on the graph represents a measurement of the voltage at a time point. After hydrogen is introduced, there is an immediate spike in voltage to roughly 900 mV. After the gas is turned off the sensor voltage returns to below 0 mV within two seconds.

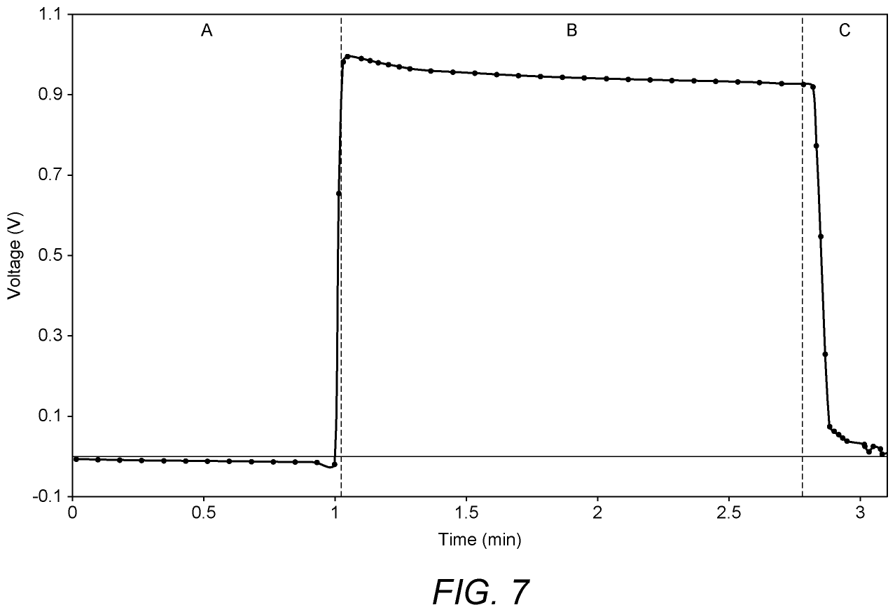

FIG. 7 shows a graph with the results from a further experiment utilising a hydrogen sensor as described herein. The graph shows voltage against time for a sensor exposed to hydrogen in one sensing event. The graph has three segments showing the three different environmental conditions: ‘A’ where the sensor is exposed to no hydrogen, ‘B’ where the sensor is exposed to hydrogen, and ‘C’ showing the sensor once again not being exposed to hydrogen, but after the exposure to hydrogen of segment B.

Each circle on the graph represents a measurement of the voltage at a time point. A voltage trace of sensor over the course of 3 minutes was recorded. The measuring side is exposed to hydrogen for just under 2 minutes, after the 1 minute time point. Prior to hydrogen introduction the voltage reading is around 0 mV. After hydrogen is introduced, there is an immediate spike in voltage to roughly 950 mV. After the gas is turned off the sensor voltage returns to around 0 mV, just before the 3 minute time point.

This demonstrates am immediate response to hydrogen exposure, with an immediate reduction back to the same sensing level as before hydrogen exposure. The graph shows that the sensing has an immediate response, is fully reversible, that there is a distinct voltage response and there is a stable voltage measurement before, during and after hydrogen exposure.

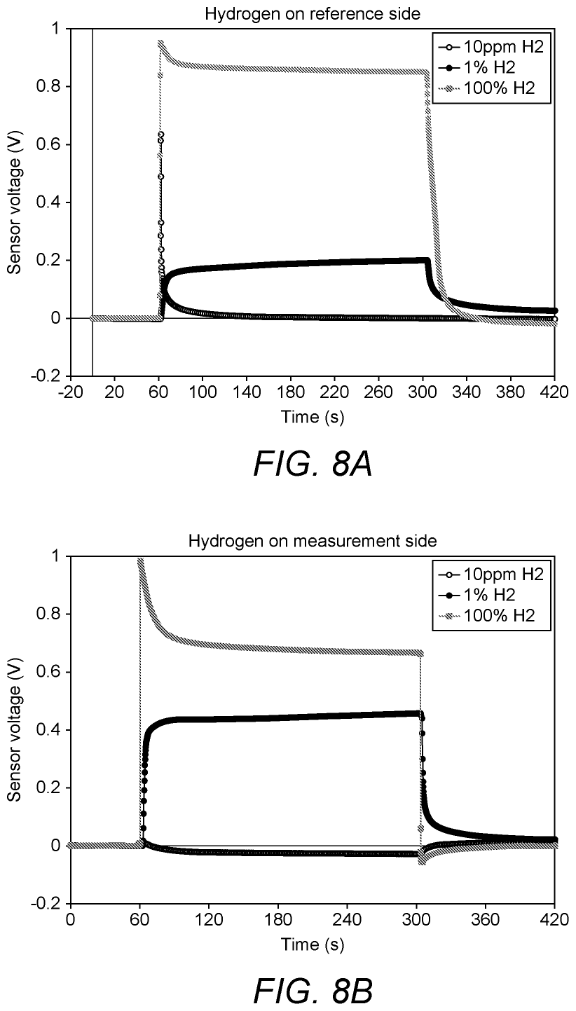

FIGS. 8a and 8b shows graphs with the results from a further experiment utilising a hydrogen sensor as described herein. One side of the sensor was exposed to hydrogen and the other side in ambient air. Two graphs of voltage against time are shown, demonstrating the sensitivity of such a sensor. Each circle on the graph represents a measurement of the voltage at a time point. Sensitivity of the sensor is demonstrated at 10 ppm, 1 vol %, and 100 vol % hydrogen, and varying sensitivity of sensor reference and measurement electrode sides of the sensor are shown. This shows that both sides of the sensor can act to sense gases, for example here hydrogen.

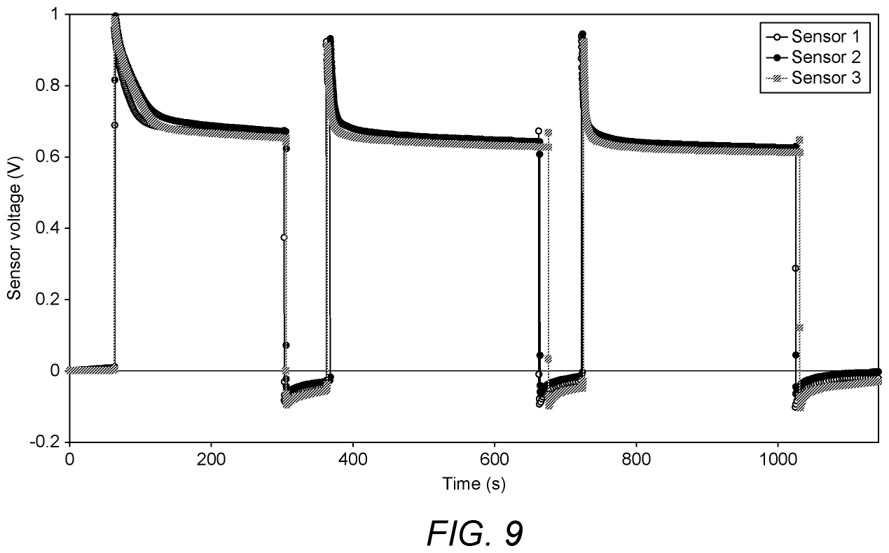

FIG. 9 shows a graph with the results from a further experiment utilising a hydrogen sensor as described herein. One side of the sensor was exposed to hydrogen and the other side in ambient air. The graph shows voltage against time but for repeated sensing events in a row. Each circle on the graph represents a measurement of the voltage at a time point.

This demonstrates that the same sensor can clearly and distinctly sense a gas, here for example hydrogen, multiple times in a row. There is no loss in sensitivity of the sensor seen across all 3 sensing events. Sensor signal is highly reproducible and fully reversible.

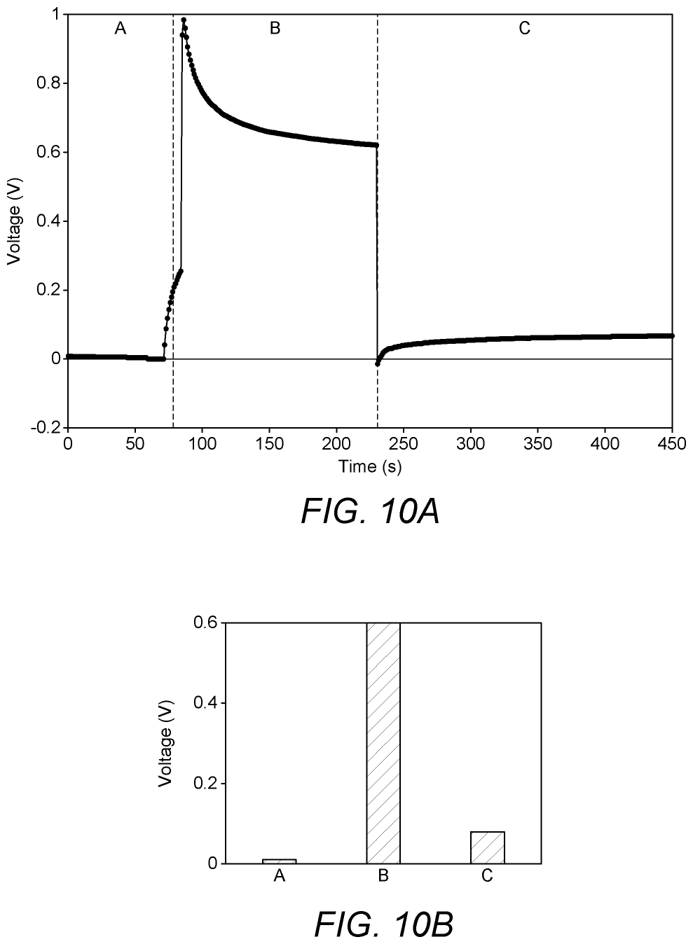

FIG. 10a shows a graph with the results from a further experiment utilising a hydrogen sensor as described herein. Each circle on the graph represents a measurement of the voltage at a time point. The graph shows voltage against time but for three different environmental conditions. The graph has three segments showing the three different environmental conditions: ‘A’ showing air on both sides of the sensor, ‘B’ showing air on the reference side and hydrogen on the sensing/measuring side of the sensor, and ‘C’ showing hydrogen on both sides of the sensor.

FIG. 10b shows voltage readings for the three different segments (A, B and C) of the graph from FIG. 10a. Segment B shows the highest reading (0.6 V). Segment C shows a reading of around 0.75V, far above the near-zero reading for segment A.

This demonstrates that even when both sides of the sensor are exposed to a gas, for example here hydrogen, the sensor is able to sense the hydrogen gas—there is still an output signal. This is due to the structure of the sensor, where one of the sides or substrates of the sensor has more openings or a larger opening than the other side or substrate of the sensor to provide increased gas volume to one side of the sensor, even when both sides are exposed to the same volume of gas at the same time. This experiment was carried out with a sensor as visible in FIGS. 1 and 2. Due to their design, sensors are sensitive to gases even when exposed to it on both sides

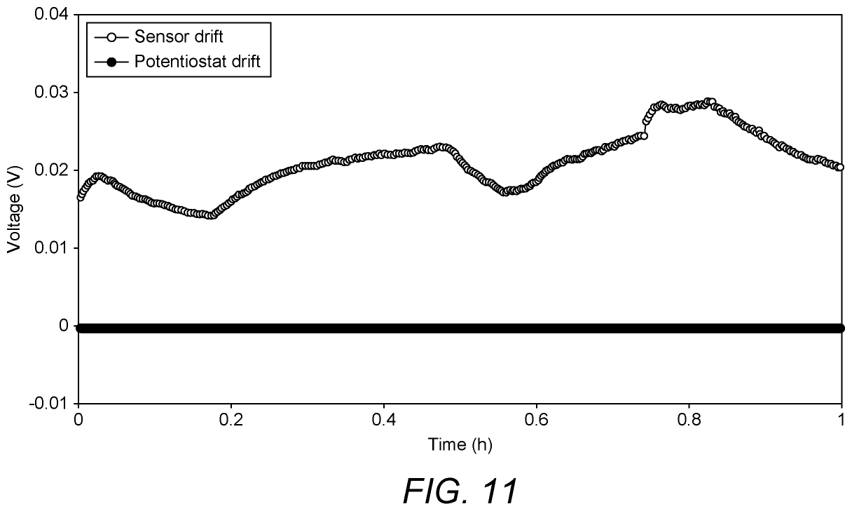

FIG. 11 shows a graph with the results from a further experiment utilising a hydrogen sensor as described herein. The graph shows voltage against time but for a sensor which was not exposed to hydrogen (above ambient air levels) for an hour. Each circle on the graph represents a measurement of the voltage at a time point.

This demonstrates that the sensors show minimal signal drift, any drift over time is many orders of magnitudes smaller than typical voltage response to hydrogen.

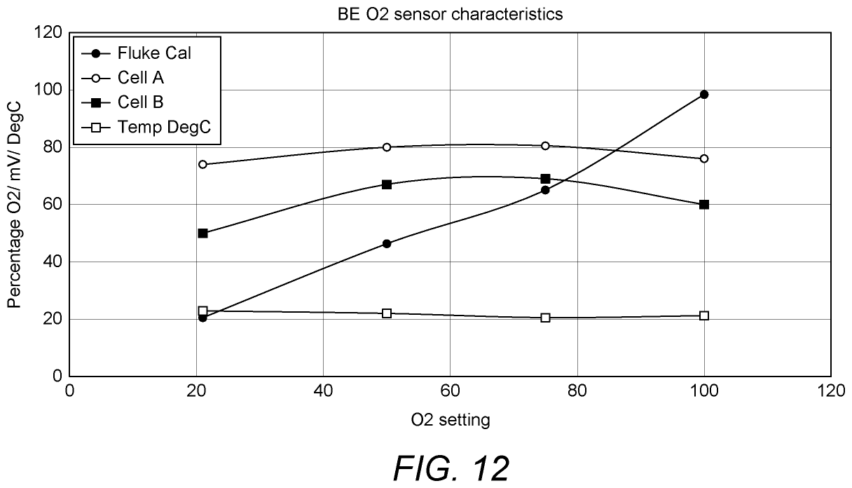

FIG. 12 shows a graph with the results from a further experiment utilising an oxygen sensor as described herein. The graph shows O₂ setting plotted against percentage O₂ The temperature is the ambient temperature during the experiments, plotted alongside the response to show if there is a need to account for sensor drift/compensate for sensor response at different temperatures.

Here two oxygen sensors ‘Cell A’ and ‘Cell B’ on the graph) were simultaneously tested against a calibrated commercially available oxygen sensor from Fluke (‘Fluke’ on the graph). Various concentrations of O₂ were tested and were in a nitrogen environment. The reference side of each sensor was exposed to the atmosphere. Sensors showed strong correlations below 70% oxygen, and sensitivity but not perfect correlation with a commercially available sensor above due to an experimental error set up. The increasing voltage of both sensors with increasing oxygen concentration (below 70%) shows a strong correlation/response of the sensor in the different environments.

The sensor used here is the same sensor as that used for the hydrogen sensing experiments demonstrated earlier.

This demonstrates that the sensor technologies described herein can be used to detect gases other than hydrogen, here oxygen. These sensors can have multiple different applications.

Thus, sensitive and accurate working sensor of the types described herein have been demonstrated.

It will be clear to one skilled in the art that many improvements and modifications can be made to the foregoing exemplary embodiments without departing from the scope of the present disclosure.

1. A fuel cell comprising:
a first sensor comprising a membrane electrode assembly (MEA) comprising a plurality of electrodes and a membrane electrolyte layer disposed between the plurality of electrodes, wherein the MEA is disposed between a first substrate and a second substrate, wherein the first substrate has at least one opening to provide a gas flow path therethrough to one of the electrodes; and
an electrical control unit configured to determine an electrical characteristic of the MEA, wherein the electrical characteristic is indicative of a gas composition of a gas at the one of the electrodes and wherein the electrical control unit will generate an output based on or in response to a change in the electrical characteristic.

2. The fuel cell of claim 1, wherein the sensor is mounted inside the fuel cell in a zone of the fuel cell where leaked gas would leak to such that any leak of reactant fuel from the fuel cell can be detected by the sensor.

3. The fuel cell of claim 1, wherein when a change in gas composition is detected by the sensor the fuel cell is turned off.

4. The fuel cell of claim 1, wherein the second substrate has at least one opening so as to provide a gas flow path therethrough to one of the electrodes.

5. The fuel cell of claim 1, wherein the fuel cell further comprises means to indicate that an output based on or in response to the electrical characteristic has been generated.

6. The fuel cell of claim 1, wherein the first substrate and the second substrate are printed circuit boards (PCBs), wherein the sensor is optionally formed by compression lamination of the PCBs.

7. The fuel cell of claim 1, wherein at least one of the first substrate and the second substrate have multiple openings to provide multiple gas flow paths through that substrate.

8. The fuel cell of claim 1, wherein one of the first substrate and the second substrate has more openings than the other of the first and the second substrate to provide more gas flow paths through that substrate to one of the electrodes than the other substrate, or wherein the opening or openings in one substrate are larger in dimension than the opening or openings in the other substrate to provide wider gas flow path(s) through that substrate to one of the electrodes.

9. The fuel cell of claim 1, wherein the MEA comprises at least two electrodes, wherein there is a gas flow path to each of the electrodes, and wherein one gas flow path is smaller or has a restricted gas flow compared to the other gas flow path.

10. The fuel cell of claim 1, wherein the electrical characteristic is potential difference, open circuit voltage between an anode side and a cathode side of the MEA, current, capacitance or conductivity.

11. The fuel cell of claim 1, wherein the electrodes further comprise a catalyst or
wherein the substrates further comprise a conductive metal layer, and the conductive metal is optionally copper.

12. (canceled)

13. The fuel cell of claim 1, wherein the fuel cell or the sensor comprises electronics capable of shorting the sensor to recalibrate the sensor.

14. The fuel cell of claim 1, wherein the electrodes are carbon paper electrodes comprising a carbon supported platinum catalyst, or
wherein the electrolyte layer is a proton exchange membrane, optionally comprising a sulfonated tetrafluoroethylene electrolyte.

15. (canceled)

16. The fuel cell of claim 1, wherein at least one of the electrodes is an electrode which will react with a fuel of a fuel source consumed by the fuel cell.

17. A sensor for detecting a change in gas composition, the sensor comprising:
a membrane electrode assembly (MEA) comprising a plurality of electrodes and a membrane electrolyte layer disposed between the plurality of electrodes,
a first substrate and a second substrate, wherein the MEA is disposed between the first substrate and the second substrate,
wherein at least one of the first substrate and the second substrate has at least one opening to provide a gas flow path therethrough to one of the electrodes; and
wherein when there is a change in the gas composition of the gas at the one of the electrodes an electrical characteristic indicative of the gas composition of the gas at the one of the electrodes can be determined to generate an output to indicate the change in gas composition detected by the sensor.

18. The sensor of claim 17, wherein one of the first substrate and the second substrate has more openings than the other of the first substrate and the second substrate to provide more gas flow paths through that substrate to one of the electrodes than the other substrate, or wherein the opening or openings in one substrate are larger in dimension than the opening or openings in the other substrate to provide wider gas flow path(s) through that substrate to one of the electrodes.

19. The sensor of claim 17, wherein the MEA comprises at least two electrodes, wherein there is a gas flow path to each of the electrodes, and wherein one gas flow path is smaller or has a restricted gas flow compared to the other gas flow path.

20. The sensor of claim 17, wherein the sensor is configured for use as a sensor in a fuel cell or as a sensor in a gas line.

21. The sensor of claim 20, wherein the one of the electrodes will react with a fuel of a fuel source consumed by the fuel cell,
wherein the sensor is located inside a housing of the fuel cell, or
wherein the output will result in turning off the normal operation of the fuel cell when a gas leak is detected.

22. (canceled)

23. (canceled)

24. (canceled)

25. A method of detecting gas or detecting a change in gas composition, the method comprising determining a change in an electrical characteristic of an MEA, wherein the change in the electrical characteristic of an MEA indicates a presence or absence of a gas or wherein the change in the electrical characteristic of an MEA indicates a change in gas composition at the MEA.
